(19) United States
(12) Patent Application Publication
(10) Pub. No.: US 2026/0337598 A1
(43) Pub. Date: Oct. 29, 2026

(54) METHOD OF SCREENING AAV VIRAL PARTICLE LIBRARIES IN ORGANOIDS

(71) Applicant: SANIA TX LTD, London, Greater London (GB)

(72) Inventors: Andrew John MURRAY, London (GB); Alba GUIJARRO BELMAR, London (GB)

(73) Assignee: SANIA TX LTD, London, Greater London (GB)

(21) Appl. No.: 19/113,372

(22) PCT Filed: Sep. 22, 2023
(86) PCT No.: PCT/IB2023/059404

§ 371 Date: Mar. 19, 2025

(30) Foreign Application Priority Data
Sep. 23, 2022 (GB) 2213924.0

(51) Int. Cl. C12N 15/10; A61K 48/00; C12N 5/077; C12N 5/0793; C12N 15/864; C40B 30/06; C40B 40/08

(52) U.S. Cl. CPC C12N 15/1058; A61K 48/005; A61K 48/0091; C12N 5/0619; C12N 5/0652; C12N 15/1082; C12N 15/8645; C40B 30/06; C40B 40/08; C12N 2750/14121; C12N 2750/14143; C12N 2750/14151

(57) ABSTRACT

The present invention relates to a method of screening AAV viral particle libraries in organoids, specifically screening for capsids that a capable of specifically infecting a population of cells, such a neurons, in preference to other cell types or populations. The invention also relates to a method of directed evolution by repeating the screening method, to the AAV viral particles obtained or evolved using the method and to a method using the AAV viral particles to infect a specific population of cells.

### FIELD OF THE INVENTION

The present invention relates to a method of screening AAV viral particle libraries in organoids, specifically screening for capsids that a capable of specifically infecting a population of cells, such a neurons, in preference to other cell types or populations. The invention also relates to a method of directed evolution by repeating the screening method, to the AAV viral particles obtained or evolved using the method and to a method using the AAV viral particles to infect a specific population of cells.

### BACKGROUND TO THE INVENTION

Adeno-associated viruses (AAV) are important vectors for gene therapies. Numerous naturally occurring AAV serotypes have been identified, each with differences in the coding sequence for the viral capsid. These differences in the capsid result in altered cellular tropism, that is, different AAV serotypes will differentially infect different cell types (Castle et al., 2016).

Naturally occurring AAV serotypes have been used in both clinical trials and in approved gene therapies. However, the prevalence of antibodies against natural AAVs in the human population, as well as the inability of natural serotypes to efficiently target certain cell types, means that synthetic (non-wildtype) capsids have become increasingly popular for genetic medicines (Kuzmin et al., 2021).

Synthetic AAV capsids have amino acids sequences that are broadly similar to naturally occurring capsids but are built in the lab via random mutagenesis, DNA shuffling or other synthetic biology techniques. Synthetic capsids can be rationally designed, for example by the insertion of peptide sequences that have potentially useful properties. Alternatively, a large, diverse library of random capsids can be generated (Korbelin et al., 2017). Typical libraries can contain up to 1012 different variants and a directed evolution approach is used to identify those variants that have desirable properties from this large pool. Desirable qualities include infection of a particular cell type of interest (Bartel et al., 2012), such as neurons, as well as detargeting from other cell types or organs that it is not desirable to target, such as the liver.

Directed evolution of AAV capsids from a library can be done in vivo or in vitro for example, by injecting the library into an animal then harvesting the target tissue or cell type (FIG. 1A). If required, this first round of evolution can be supplemented with further rounds, whereby the harvested capsid DNA is further mutated or modified, and the screening process repeated (Grim and Bueng, 2017).

As is generally the case with directed evolution approaches “you get what you screen for”. That is to say, the closer that your screening environment replicates the final targeted system, the more likely it is that you will find candidates that possess the desired properties (Schmidt-Dannert and Arnold. 1999). For gene therapy approaches, it is therefore critical that the screening of AAV capsid libraries occurs in a system that resembles the final human patient as closely as possible.

Directed evolution of AAV capsids often occurs in experimental animals, such as mice or non-human primates (NHP). Mice are favoured due to their widespread availability in experimental research and ease of genetic manipulation, whereas NHP are evolutionarily closer to humans. In both cases, the capsid libraries are injected into experimental animals, the tissue or cell type of interest harvested after an incubation period, and the AAV particles recovered are then sequenced to identify useful capsids. As expected, capsid evolution that occurs in mice results in capsids that are effective in mice but not in NHP (Ligoure et al., 2019), with capsid properties also being tied to the precise strain of mouse used (Mathieson et al., 2020). Consequently, the most effective capsid evolution strategies, at present, use a combination of animal screening and validation on human cells (see Tabebordbar et al., 2021).

Organoids can be defined as a 3D structure, grown from stem and progenitor cells (induced pluripotent stem cells, IPSCs; embryonic stem cells, ESCs) and consisting of organ-specific cell types. These cell types self-organise through cell sorting and spatially restricted lineage to form a representation of specific (human) tissue “in a dish” (Bredenoord et al., 2017). Numerous different types of organoids have been developed for a variety of human tissues, including: parts of the gastric system, small intestine, colon, liver, pancreas, trachea, pulmonary alveolus, thyroid, oesophagus, prostate, fallopian tube, kidney, and various parts of the nervous system such as: the retina, cerebellum, cerebrum, olfactory bulb, hippocampus, hypothalamus, choroid plexus, spinal cord and neuromuscular system (see Rossi et al., 2018).

A number of studies have used organoids to test whether previously identified capsids (either synthetic or wildtype capsids) would be efficacious in human tissues. For example, Achberger et al. (2021) recently used retinal organoids to test the tropism of seven different AAV vectors (both wildtype and synthetic) to assess which had the best tropism, efficacy and kinetics in human tissue. Similarly, Garita-Hernandez et al. (2020) used retinal organoids to test the efficacy of several AAV serotypes in human retinas, and McClements et al. (2022) used retinal organoids to evaluate the effectiveness of both AAV capsids and promoters in gene expression in photoreceptors. Depla et al. (2020) used human cerebral organoids to test the efficacy of two AAV serotypes, AAV5 and AAV9, in a human context. They found that AAV5 provided superior transduction.

Outside of the nervous system, human organoids have been used to test the ability of different AAV serotypes to infect the lung (Meyer-Berg et al., 2020) and to screen six different synthetic capsids for their ability to infect cell types in kidney organoids (Ikeda et al., 2018).

The above studies represent the use of human organoids in the testing of AAV capsids which have previously been identified by other means. Importantly, they demonstrate the belief that human tissues in vitro will provide a superior environment for testing of human therapeutics than animal models. However, there are no studies, or descriptions, of using human tissues or organoids as a vehicle to search for and identify novel AAV capsids.

The present disclosure relates to a method of screening AAV capsids using organoids to select for a capsids that selectively infect a particular cell type and to a method of directed evolution using the same organoid. The method presents the first step in identifying novel vectors for gene therapy.

### SUMMARY OF THE INVENTION

According to a first aspect there is provided a method of screening for capsid-encoding nucleotide sequences of AAV viral particles capable of infecting population-specific neurons, the method comprising:
(i) obtaining or having obtained a neuromuscular organoid comprising neurons and muscle cells, wherein the neurons, having a cell body and a distal portion located away from the cell body, are disposed in the organoid such that the cell body and the distal portion are distally separated from each other;
(ii) contacting the distal portion of the neurons with a population of AAV viral particles such that they can infect the neuron at the distal portion;
(iii) recovering AAV viral particles that have infected the distal portion of the neurons from the cell body; and
(iv) determining the capsid encoding nucleotide sequences of the AAV viral particles recovered from the cell body.

In a second aspect there is provided a method of directed evolution to select for AAV viral particles capable of selectively infecting population-specific neurons comprising the steps:
(i) obtaining or having obtained a neuromuscular organoid comprising neurons and muscle cells, wherein the neurons, having a cell body and a distal portion located away from the cell body, are disposed in the organoid such that the cell body and the distal portion are distally separated from each other;
(ii) contacting the distal portion of the neurons with a population of AAV viral particles such that they can infect the neuron at the distal portion;
(iii) recovering AAV viral particles that have infected the distal portion of the neurons from the cell body;
(iv) determining the capsid encoding nucleotide sequences of the AAV viral particles recovered from the cell body, and
(v) optionally using the output of step (iv) to generate a new AAV viral particle library and repeating the method.

In one embodiment the neuromuscular oganoid is derived from iPSCs or ESCs.

In one embodiment the population-specific neurons are selected from the list consisting: mammalian, human, human sub-population and human disease-specific.

In one embodiment the human disease-specific population-specific neurons are selected from the list: MND, DM, ALS, HD, epilepsy, neuropathy, PD, SCA, HSP, PLS, SMA, SBMA and LCCS neurons.

In a third aspect there is provided AAV viral particles capable of infecting population-specific neurons identified or selectively evolved according to the method disclosed herein.

In a fourth aspect there is provided the use of the AAV viral particles according to the present disclosure to selectively infect population-specific neurons.

In one embodiment the AAV viral particle comprises a nucleotide-encoded payload.

In one embodiment the nucleotide-encoded payload encodes a therapeutic peptide.

In one embodiment the nucleotide-encoded payload is a gene therapy payload.

In a further aspect there is provided the use of the AAV viral particles disclosed hereing in the treatment of a disease caused by a genetic mutation.

The invention provides an AAv capsid identified by a screening method of the invention.

Advantageously, the AAV capsids identified by the screening methods described herein may be used to develop gene therapies for treating various conditions or disorders. Accordingly, the AAV capsids may be employed in a method of ameliorating or treating a neuromuscular or neuromotor condition or disorder in a subject, comprising administering to the subject a therapeutically active amount of an AAV expression vector or viral particle of the invention.

### BRIEF DESCRIPTION OF THE DRAWINGS

For a better understanding of the invention and to show how the same may be carried into effect, there will now be described by way of example only, specific embodiments, methods and processes according to the present invention with reference to the accompanying drawings in which:

FIG. 1A shows a prior art method of directed evolution of AAV capsids in which an AAV genome capsid library is generated that contain a wide range of capsid variants and packaged into an AAV vector capsid library. The library is injected into an experimental animal (19) and then capsids/AAVs are harvested from target tissues (20) and sequenced.

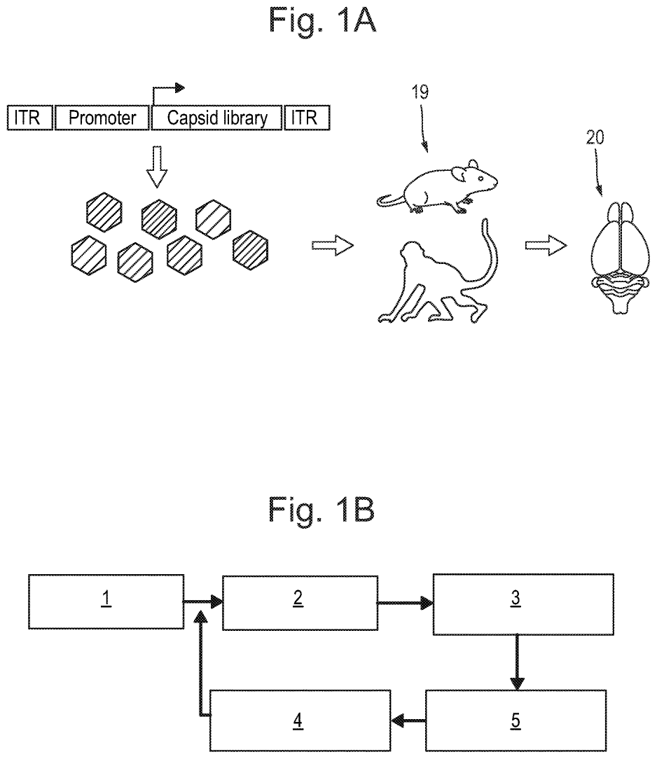

FIG. 1B shows a flowchart of the method of identification of AAV capsid variants from an organoid in which 1—generation of tissue specific organoid, 2—infection of organoid with AAV capsid library, 3—harvesting of capsid DNA or RNA from organoid cell type of interest, 5—sequencing and analysis of enriched variants and 4—production of new library.

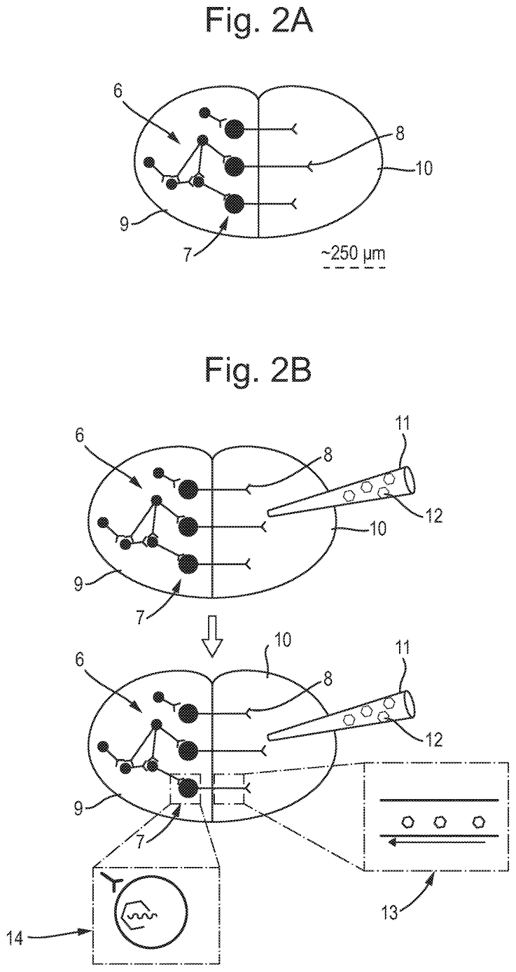

FIG. 2 shows an example use of neuromuscular organoids to screen for motor neuron targeted AAV capsids.

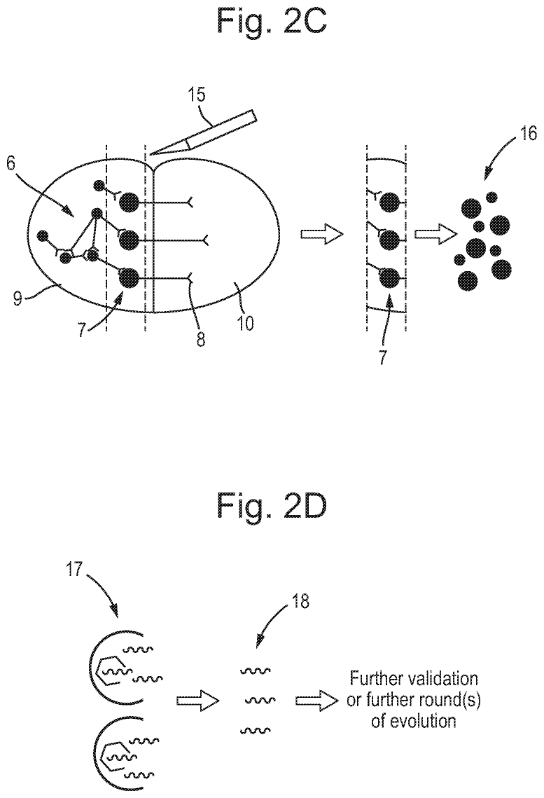

2A shows a spinal-neuromuscular organoid with a spatially separated spinal cord compartment (9) containing interneurons (6) and motor neurons (7) and an innervated muscle compartment (10) containing motor neuron axons and synaptic terminals (8).

2B shows microinjection (11) of the AAV capsid library (12) into the muscle compartment, some members of the library infect the motor neuron synaptic terminal and are retrogradely transported within the motor neuron axon (13) to the motor neuron cell body (14) within the spinal cord compartment and release of the capsid DNA occurs.

2C shows microdissection and harvesting of the motor neurons (7) and dissociation of the cell bodies (16) from the rest of the tissue.

2D cells are lysed (17) to release capsid DNA and this DNA (18) is then sequenced to identify capsids that have infected motor neurons. These capsids can then be further validated and subjected to additional rounds of evolution

### DETAILED DESCRIPTION

As employed herein screening refers to a method of selectively identifying members of a population for desirable properties. Herein, the population is an AAV library, or AAV viral particle library and the desirable property is the ability to selectively infect population-specific cells, such as population-specific neurons.

As employed herein capsid-encoding nucleotide sequences refers to nucleotide sequence encoding the capsid proteins. Typically, the nucleotide sequence is a DNA sequence, although RNA, modified nucleotide and synthetic nucleotides are also envisioned.

As employed herein AAV viral particles refers to adeno-associated virus viral particles comprising a single stranded DNA genome packaged within a viral envelope that is capable of infecting a cell, e.g. a mammalian cell. In particular, the AAV viral particles employed herein include the nucleotide sequence for the capsid that is packaging the nucleotide sequence. In some embodiments the viral particles may be devoid of replication-encoding nucleotides, that is, they are replication deficient.

AAVs are small viruses belonging to the genus dependoparvovirus containing a single strand of DNA, up to ^{~}4.9 Kb. The AAV genome contains three capsids proteins VP1, VP2 and VP3, all of which are translated from one mRNA via alternate splicing. In the wild, multiple serotypes of AAV have been identified each with unique sequences of capsid gene, and hence distinct tropisms, although wild serotypes tend to be able to infect multiple tissue and cell types. These serotypes are denoted by numbers: AAV1, AAV2, etc. It has been shown that modification of capsid sequences via DNA recombination methods can generate non native sequences with tailored properties and tropism directed towards (or against) particular cells or tissues, and that evade the immune system (Vandenberghe et al., 2009).

Modifications to capsids can be achieved in several ways, such as random mutagenesis of existing capsid DNA sequences, by capsid shuffling (i.e. taking DNA sequences from multiple capsids and randomly shuffling parts of the sequence to make a new capsid; Buning et al., 2015), or by insertion of short peptide sequences into the exposed loop regions of the capsid. These methods can generate a large number of highly diverse capsids with potentially valuable properties. When packaged into functional virions, they can be screened in animal tissues or in cell cultures to select those capsids that are targeted towards particular tissues or cells. These capsid sequences can then be generated de novo and combined with genes with therapeutic potential in a gene therapy.

As employed herein “capable of infecting population-specific neurons” refers to the ability to selectively infect a particular cell type such as neurons rather than muscle tissue.

However, population-specific may further refer to selectively infecting, for example, animal neurons or human neurons, such as human neurons rather than rat neurons.

Further, the method described herein may be used to identify viral particles capable of infecting a sub-population of neurons, for example, disease-state human neurons rather than non-disease-type neurons. Such as Duchenne muscular dystrophy (DM), Amyotrophic lateral sclerosis (ALS), Epilepsy, Huntington disease (HD), neuropathy, Parkinson disease (PD), Spinocerebellar Ataxia (SCA), hereditary spastic paraplegia (HSP), primary lateral sclerosis (PLS), spinal muscular atrophy (SMA), spinal bulbar muscular atrophy (SBMA) and lethal congenital contracture syndrome (LCCS) or other genetic disorders.

Furthermore, population-specific may relate to the ability to selectively infect one part of a neuron, such as the axon or dendrite, rather than the cell body.

As employed herein, the term neuron includes a neuron and a portion or portions thereof (e.g., the neuron cell body, an axon or a dendrite). The term neuron as used herein denotes nervous system cells that include a central cell body (or soma) and two types of extensions or projections: dendrites, by which the majority of neuronal signals are conveyed to the cell body, and axons, by which the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal cord). Yet more neurons, designated project (or “projection”) neurons, extend their axons from one region of the nervous system to another. The screening methods disclosed herein may be used to screen for capsid-encoding nucleotide sequences of adeno-associated virus (“AAV”) particles capable of infecting such neurons.

It will be appreciated that the method disclosed herein may be applicable to cell types other than neurons.

In some embodiments the population-specific neurons are motor neurons.

In other embodiments the population-specific neurons are sensory neurons

In some embodiment the population-specific neurons are interneurons (or genetically defined subtypes).

In some embodiments the population-specific neurons are projection neurons (or genetically defined subtypes).

In one embodiment the population-specific neurons are human neurons.

In one embodiment the population-specific neurons are disease-state human neurons.

As employed herein disease-state neurons means neurons or neuronal subtypes implicated in the pathophysiology of a particular disease. For example, dopaminergic neurons in Parkinson's disease or motor neurons in amyotrophic lateral sclerosis etc.

As employed herein organoid refers to a miniaturised and simplified version of an organ produced in vitro in three dimensions that shows realistic micro-anatomy. They are derived from one or a few cells from a tissue, embryonic stem cells or induced pluripotent stem cells, which can self-organise in three-dimensional culture owing to their self-renewal and differentiation capacities. In the example of neuromuscular organoids, these comprise of both muscle and neurons and may comprise both motor-neurons and spinal cord. Neuromuscular organoids are described, for example, in Martins et al. (2020), Periera et al. (2021), and Andersen et al. (2020). Other types of nervous system organoid are envisioned in the current invention, such as retinal, cerebellum, cerebrum, spinal cord etc.

In one embodiment the organoid is derived From IPSCs or ESCs.

In one embodiment the organoid is a human organoid.

In one embodiment the organoid is a human disease-state organoid.

In one embodiment the organoid is a neuromuscular organoid.

As employed herein, a disease-state organoid is an organoid grown from cells or tissues known to carry a genetic mutation that results in a pathology.

Distally separated as employed herein means that the parts of the neuron are located in physically distanced parts of the organoid such that viral particles can be delivered to a part of the neuron, such the axon or dendrite, in preference to another, such as the cell body. Advantageously, by recovering only viral particles that are found, for example, in the cell body, or spinal cord it is possible to determine which particles can infect neurons.

Distal portion as employed herein refers to parts of the neuron that are distal to the cell body, such as the axon and dendrites, particularly the axon.

Without wishing to be bound by theory, it is thought that some AAV particles may target receptors specifically located at the axon and infect the neuron using these receptors.

As employed herein, “contacting the distal portion of the neurons with a population of viral particles such that they can infect the neuron at the distal portion” refers to any method of exposing the distal portion of the neuron to the viral particles. This can include, but is not limited to injection, microinjection into a particular compartment of the organoid (such as the muscle compartment of a neuromuscular organoid) or inclusion of AAV vectors in the culture media.

As employed herein “recovering viral particles that have infected the distal portion of the neurons from the cell body” refers to any method of selectively recovering only viral particles that have successfully infected the neuron by harvesting the cell body. This can include, but is not limited to, harvesting of a component of the culture, such as microdissection of the spinal cord compartment in a neuromuscular organoid, or harvesting of targeted cell types via somatic inclusion of a fluorophore in the targeted cell type followed by dissociation of the organoid and fluorescent activated cell sorting (FACS). As employed herein “determining the capsid encoding nucleotide sequences of the viral particles recovered from the cell body” refers to sequencing of the viral particle nucleotide sequence of those viral particles recovered from the cell body, typically using high throughput sequence of the type known in the art, such as next generation sequencing (NGS).

Advantageously, the nucleotide sequences of viral particles harvested from the cell body can be compared to those of either the library as a whole or to another source, such as those gathered from muscle cells in the neuromuscular organoid or separate classes of neuron, for example. Comparison of the differences between these sequences enables trends to be identified and further AAV libraries to be rationally designed.

Advantageously, the viral nucleotide sequences can then be analysed and used to generate new libraries that may be used in the screening method described herein to provide in a novel directed evolution method.

In vivo studies have shown that directed evolution of AAV capsids can lead to vectors that have useful properties such as an ability to cross the blood brain barrier and increased neurotropism (Deverman et al., 2016 and EP3044318B1), or directed towards dopaminergic neurons (Daviddson et al., 2019) or cardiomyocytes (Yang et al., 2009) amongst others (for review see Li and Sumulski, 2020). Examples of directed evolution include the generation of a capsid library, a mixture of AAV vectors encapsidated with random capsid sequences generated via error prone PCR, capsid shuffling, or other methods. These libraries have been applied to cell lines (e.g. Maheshri et al., 2006), undifferentiated stem cells (Asuri et al., 2012) or, most commonly, in experimental animals (for examples Devermann et al., 2016; Li and Sumulski, 2020; Patents U.S. Pat. No. 8,632,764B2; US20170166926A1 ; U.S. Pat. No. 9,701,984B2). The present invention advantageously provides a method of directed evolution utilising organoids.

Population-specific AAV viral particle as employed herein refers to an AAV viral particle that selectively infects a population-specific neuron, such as an AAV viral particle identified or evolved using the method disclosed herein.

As discussed above, AAV capsids have the potential for exquisite levels of cell and species tropism but currently available screening technologies fail to fully harness this selectivity and utilise it for therapeutic benefit.

The present invention overcomes these drawbacks and allows for the screening of AAV capsid libraries in organoids derived from induced pluripotent stem cells (“iPSCs”) or embryonic stem cells (“ESCs”). Organoids can be derived in vitro from iPSCs or ESCs (Little et al., 2019). As the genetic makeup of these organoids would be more similar to that of human patients than the same cells found in animal models, it is believed that they provide a much more suitable substrate for screening AAV libraries than experimental animals. In some embodiments, the organoids are neuromuscular and the neurons are motor neurons.

The present invention also comprises a viral evolution approach that uses a combination of organoids derived from induced iPSCs and ESCs, and in vitro screening, to identify AAV capsid-encoding nucleotide sequences that allow for AAV capsids that can more efficiently infect neurons, specifically population-specific neurons. In some embodiments, this approach allows for the identification of AAV capsid-encoding nucleotide sequences that allow for AAV capsids that can more efficiently infect motor neurons via intramuscular injection.

The invention also comprises a technology platform that identifies novel adeno-associated virus (AAV) capsids based on their ability to target population-specific neurons, by using iPSC/ESC-derived organoids. An example use of this is described herein, where AAV capsid libraries can be screened for their ability to efficiently infect population-specific neuron terminals, but the same system may be used to identify capsids targeted to many neuronal types.

Further, as capsid sequences that efficiently infect neurons may vary from subject to subject, the invention further comprises a process whereby skin samples from individual subjects can be taken, the skin sample transformed into stem cells and then organoids, and these organoids from the patient are used to screen for effective AAV vectors, providing a personalised approach to gene therapy.

In some cases, the iPSCs or ESCs are derived from the subject. In some cases, the iPSCs or ESCs are derived from a skin sample of the subject. In some cases, the iPSCs or ESCs are derived from fibroblasts of the subject. In some cases, the subject is a human subject. The iPSCs and ECSs may also be obtained from an animal, a human subject/patient, or from a cell bank. The screening of AAV capsid libraries using iPSCs from a human subject or patient allows for the identification of capsid sequences that infect neurons or other cells from that subject or patient. Accordingly, the capsid sequences can be generated on an individualised basis using this method.

In some cases, the method is a method a screening for capsid-encoding nucleotide sequences of adeno-associated virus (“AAV”) particles capable of infecting neurons in a subject via intramuscular injection.

In one embodiment the organoids are derived from iPSCs.

In one embodiment the organoids are derived from ESCs.

In some embodiments the iPSCs used in the screening methods described herein are derived from a human test subject.

In some embodiments the iPSCs are derived from a culture of fibroblasts from skin biopsies. Some methods that allow for culturing of fibroblasts from skin biopsies have been previously described (Vangipuram M, Ting D, Kim S, Diaz R, Schule B. Skin punch biopsy explant culture for derivation of primary human fibroblasts. J Vis Exp. 2013; (77): e3779. Published 2013 Jul. 7. doi:10.3791/3779).

In some embodiments the iPSCs used in the screening methods described herein are derived from a human test subject.

In some embodiments the iPSCs are derived from a culture of fibroblasts from skin biopsies of the subject.

Derivation of human fibroblasts into iPSCs can be achieved using a commercial kit such as the CytoTune-IPS Sendai reprogramming kit from ThermoFisher. Further details may be found at https://www.thermofisher.com/order/catalog/product/A16517#/A16517 and https://assets.thermofisher.com/TFS-Assets/LSG/manuals/cvtotuneips20sendaireproqkitman.pdf.

Recombinant AAV (rAAV) vectors developed using the methods described herein are especially applicable to the treatment of neuromuscular/neuromotor disorders such as spasticity, amyotrophic lateral sclerosis, dystonia, allowing for the introduction of genetic material into motor neurons via intramuscular injection of viral vectors. However, the screening methods defined herein are agnostic to the disease being treated and may be used to screen for capsid sequences that effectively target many neuronal types, and as such could be effective in gene therapy approaches regardless of the neuron disorders (or “neuronal disorders”). It is believed the invention may also be applied to other neuron disorders which currently have little or no effective therapies, as well as to other types of neuron that underlie neurological disorders.

The effectiveness of an AAV particle to infect population-specific neurons can be determined by counting the number of neurons that express the viral DNA.

For example, multiple motor neurons innervate the same targets, e.g. muscle in the case of motor neurons, and the proportion of these neurons that have been transduced by the AAV viral particle can be counted. The effectiveness of an AAV viral particle to infect neurons or specific parts of neurons can also be determined by DNA sequencing or RT-PCR to look at the “copy number” of the viral DNA that is in the neural cell. This would give an estimate of how many times the same cell was infected with the AAV viral particle.

The application of AAV capsid libraries to a distal portion of a neuron (such as the axon, dendrite, neurite, or axon terminator “synaptic terminal”) and harvesting genetic material from another region of the cells (such as the cell body) allows for the identification and determination of AAV capsid sequences that have successfully infected those cells via that specific route.

It is believed that the screening methods described herein may be applied to screen for capsid-encoding nucleotide sequences of AAV viral particles capable of infecting other cell types such as sensory neurons, basal ganglia projection neurons, dopaminergic neurons and muscle tissue. Advantageously, this allows the identification of capsid sequences that infect other cell types and allows the capsid to be design away from those sequences, thereby reducing the likelihood of cross-infectivity.

In some cases, the screening methods described herein comprise a step of providing a plurality of test AAV viral particles that have been additionally screened for capability of infecting muscle cells, for example by direct intramuscular injection. Said additional screening may occur before, after or concurrently with screening of the particles for capability of infecting neurons. Advantageously, comparative data of this type permits selection of capsids that target the desired cell types.

In some cases, the screening method can make use of multiple organoids in a single culture system. For example, organ on a chip or organoid fusion technology (Kakni et al., 2022) can be used to maintain multiple organ systems in one in vitro system. For example, dorsal and ventral forebrain organoids can be fused to produce interacting spheroids (Birey et al., 2017); assembled thalamic and cortical organoids contain reciprocal thalamocortocal and corticothalamic organoids (Xiang et al., 2019) and multiple organ systems such as liver, heart and lungs can be integrated into a single chip to study interaction, and in the present example AAV capsid infectivity, across multiple systems (Skardal et al., 2017). The use of multiple organoids in a single culture system would provide a screening environment closer to full human systems and further increase the ability to identify AAV capsids than can target one organ or cell type over another.

The screening method described herein requires generation of AAV viral particle libraries, or capsid libraries. Methods of generating libraries are known in the art. One such method is described in Nonenmacher et al., 2021.

Diverse capsid libraries can be generated through a process of, for example: i) random mutagenesis of naturally occurring capsids, ii) shuffling of naturally occurring capsids, iii) insertion of targeted or random peptide sequences up to 25 amino acids in length at various regions in VP1, VP2 or VP3 of the AAV capsid or iv) a combination of the above.

To generate the library the randomised capsid sequences are cloned into an AAV backbone containing the AAV2 inverted terminal repeats (ITRs; packaging signals) and the AAV rep gene. These DNA plasmids are transfected in to HEK293 in the presence of additional adenoviral genes to facilitate AAV packaging. AAV virions are harvested from HEK293 cells and/or the culture medium, purified and concentrated following standard methods (e.g. Potter et al., 2014 https://dx.doi.ora/10.1038%2Fmtm.2014.34; McClure et al., 2011 htp://dx.doi.Org/10.3791/3348)

In some embodiments, methods for production of AAV libraries comprise one more of the following steps:
Purified and concentrated AAV libraries are diluted in Dulbecco's modified Eagle medium (DMEM) and this is applied to the muscle chamber of a microfluidic device.
2-10 days after application neuronal cell bodies are harvested either by chemical (i.e. trypsinisation) or mechanical (cell scraping) methods.
The neurons are lysed and the lysate can either be submitted for deep sequencing (such as RNAseq or DNAseq) to directly detect capsid sequences found in the neurons, or the lysate can be used as a PCR template using primers directed against conserved regions of the AAV capsid. Following PCR of capsid regions the DNA fragment is cloned into a DNA vector and submitted for Sanger sequencing.
Capsid sequences harvested from neurons are analysed using bioinformatics for conserved regions and highly enriched capsids can be de novo synthesised and can then undergo either further mutagenesis or repeat of in vitro screening to increase evolutionary pressure through directed evolution.
Directed evolution can be repeated for several rounds (^{~}2-5 rounds). Capsid sequences that show efficient retrograde transport in vitro can be used to generate functional virions for in vivo use

In one embodiment the viral particles that selectively infect population-specific neurons (i.e. population-specific AAV viral particles) are used to deliver a nucleotide-encoded payload or exogenous transgene to the population-specific neurons following infection. Advantageously, this payload can be used to deliver gene therapy to targeted cells.

In one embodiment the population-specific AAV viral particles can be used as viral vectors, such as expression vectors. Such expression vectors can be used in gene therapy methods to deliver exogenous transgenes to a subject in need thereof.

Typically, the gene therapy will be used to treat a neurological disorder or condition. One example of a neurological disorder or condition that can be treated by the gene therapy is spasticity. Spasticity is a neurological symptom suffered by people with a variety of neurological disorders, including but not limited to multiple sclerosis, stroke, traumatic brain injury, spinal cord injury, and cerebral palsy. Spasticity results from excessive excitation of muscle by motor neurons, which, because of the disease, become “hyperexcitable.”

The invention also comprises methods of treatment which involve injecting AAVs comprising the novel population-specific AAV capsids identified using the screening or directed evolution method disclosed herein into affected muscles of a subject; these AAVs can then infect the distal portion of motor neurons, and are transported to their cell bodies leading to the expression of an exogenous transgene specifically in population-specific neurons innervating that muscle, providing high specificity.

The invention can therefore enable the generation of viral vectors (or expression vectors) that access population-specific neurons and subsequently modify gene expression in the population-specific neurons with the goal of curing, alleviating symptoms, and/or improving the quality of life of patients with diseases affecting, for example, motor neurons.

The invention also comprises methods of treatment which involve injecting AAV viral vectors comprising the novel population-specific AAV capsids into the brain or spinal cord of a subject; these AAV viral vectors can then infect the distal portion of motor neurons, sensory neurons, interneurons or projection neurons of a subject and may selectively infect the distal portions of those neurons in a specific population.

The invention can therefore enable the generation of viral vectors that access these neurons following intracranial, intraspinal or intramuscular injection, and subsequently modify gene expression in these neurons with the goal of curing, alleviating symptoms, and/or improving the quality of life of patients with diseases affecting these neurons.

The capsid nucleotides identified by the present method may be used to develop gene therapies involving viral vectors that access neurons following intramuscular injection, and subsequently modify activity and/or gene expression in neurons. Said nucleotides may also be used to develop gene therapies involving viral vectors that access neurons following intramuscular injection, and subsequently modify activity and/or gene expression in neurons.

In some embodiments, the viral vector is capable of altering the activity of targeted population-specific neurons in a subject. In some embodiments, the viral vector is capable of altering the activity of targeted motor neurons in a subject via intramuscular injection.

In some embodiments, the viral vector further comprises a transgene encoding a transgene product, wherein the transgene product is capable of altering the activity of targeted population-specific neurons in a subject. In some embodiments, the transgene product is capable of altering the activity of targeted population-specific neurons in a subject via intramuscular injection.

In some embodiments methods for the addition of a payload to alter neuron activity may include one or more of the following steps:
Capsid sequences identified in the screening methods described herein are de novo synthesised and inserted into an AAV helper plasmid containing the AAV2 REP gene (Rep/Cap).
The Rep/Cap plasmid is combined with the AAV backbone and additional plasmids containing adenoviral helper genes (such as pHelper) and transiently transfected into HEK293 cells.
AAV particles are purified using standard methods and can be used for experiments in vitro or in vivo (e.g. Potter et al., 2014 https://dx.doi.org/10.1038%2Fmtm.2014.34: McClure et al., 2011 http://dx.doi.org/10.3791/3348).

The invention also provides AAV viral particles, for example those produced by the methods described herein.

In some embodiments, the AAVs capsids will be “individualised” to the subject, and the method of treatment will be specific (“personalised”) to the subject being treated by generating a personalised organoid.

In some embodiments, the method involves the AAV expression vector or viral particle retrogradely infecting neurons for the purposes of delivering genetic material to neurons, with the purpose of treating neuromuscular or neuromotor disorders, or disorders affecting movement or targeting neurons for any therapeutic purpose.

In some embodiments, the AAV expression vector or viral particle is delivered intramuscularly, in order to infect motor neurons of a subject neuron retrogradely and alter the activity of the motor neurons in a subject.

As used herein, “retrograde transport” or “retrograde infection” means uptake of the vector at the axon terminal (or “synaptic terminal”), i.e., at the synaptic portion, and transport through the axon in a direction opposite to the direction of propagation of action potentials (and thus “retrograde”) and into the body of the neuron. Subsequently, the viral nucleic acid can enter the nucleus where it can be replicated and become transcriptionally and translationally active.

Such delivery is advantageous when the neuronal cell body and or axon themselves are inaccessible, but their terminal projection fields including synapses, are available for delivery of the genetic vector.

Successful delivery to such a terminal projection field of a genetic vector capable of retrograde transport would thus result in retrograde transport and infection of the vulnerable projection neurons.

Once the viral vector is transported to the body of the neuron, the viral nucleic acid typically localises to the nucleus of the cell. According to some embodiments of the present invention, adeno-associated viral particles that undergo retrograde transport to the neuronal body can insert their nucleic acid content directly into the nucleus.

Embodiments of the invention involve delivery of a substantially non-toxic, recombinant adeno-associated virus vector having a heterologous gene of interest in order to provide retrograde gene delivery to a neuronal cell body resulting in gene expression.

In some embodiments, AAV expression vectors of the invention are capable of treating a neuromuscular or neuromotor disorder by accessing motor neurons following intramuscular injection, and subsequently modifying gene expression in motor neurons, leading to curing, alleviating symptoms, and/or improving the quality of life of patients with diseases affecting motor neurons.

As employed herein, neurological disorder refers to a disorder which causes morphological and/or functional abnormality of a neural cell or a population of neural cells. The neurological disorder can result in an impairment or absence of a normal neurological function or presence of an abnormal neurological function in a subject. For example, neurological disorders can be the result of disease, injury, and/or aging. Non-limiting examples of morphological and functional abnormalities include physical deterioration and/or death of neural cells, abnormal growth patterns of neural cells, abnormalities in the physical connection between neural cells, under-or over production of a substance or substances, e.g., a neurotransmitter, by neural cells, failure of neural cells to produce a substance or substances which it normally produces, production of substances, e.g., neurotransmitters, and/or production or transmission of electrical impulses in abnormal patterns or at abnormal times. As used herein, a “neuromotor disorder” is a developmental or acquired disorder that typically affects movement/gross motor ability, posture, and fine motor ability. The disorder is caused by damage to the central nervous system. This could be due to problems with development or injury to the developing motor pathways in the cortex, basal ganglia, thalamus, cerebellum, brainstem, spinal cord, or peripheral nerve. The most common neuromotor disorders in childhood include cerebral palsy, muscular dystrophy, and spina bifida. The most common neuromotor disorders in adults include stroke, multiple sclerosis, Parkinson's disease and traumatic injury. The impairment may be static (not getting worse) or progressive.

The term “treatment” also includes combination treatments and therapies, in which two or more treatments or therapies are combined, for example, sequentially or simultaneously.

The invention also provides a cell comprising an AAV viral vector as described herein. In some embodiments, this cell is a mammalian cell such as a human cell.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

### EXAMPLES

### Example 1—AAV Capsid Screening with Organoids Growing in Microfluidic Chambers

1. hiPS Cell Culture and Differentiation
a. The iPS cells were cultured on a Vitronectin-coated dish with Essential 8 Medium to maintain the undifferentiated state.
b. hiPSCs were dissociated with Accutase, seeded on Matrigel-coated dish with Essential 8 Medium with ROCK inhibitor, and cultured until they reached 80% confluence. hiPSCs were then dissociated with Accutase and resuspended in differentiation medium N2B27 and cultured in non-adherent, ultra-low attachment.
c. At Day 2, Embriod Bodies are formed and were transferred to a new dish and cultured in N2B27 with Chir-99021.
d. At day 4 Retinoic acid and Smoothened Agonist were added.
e. At day 9, DAPT was added.
f. From Day 11 onwards, BDNF and GDNF were added to differentiation media and media was changed every other day.

2. Formation of Motor Nerve Organoids
a. The differentiated cells were dissociated by Accutase and seeded in a low-adhesion V-bottom 96-well plate for 10 days to generate spheroids.
b. Microfluidic devices were coated with Matrigel and the spheroids were transferred into one side of the microfluidic chamber. Half of the culture medium was replaced with fresh medium every 4 days.
c. hIPSCs derived myoblasts were plated at the other side of the microfluidic
d. Typically, 20-30 days after spheroids are transferred into the microfluidic chamber, an axon fascicle forms within the microgrooves connecting with the muscle cells growing at the other channels.

3. AAV Capsid Libraries Addition
a. Libraries were added to the side where muscle cells are growing and forming connections with the axonal terminal.
b. Media was changed to the muscle side 8 hours after libraries were added to ensure fluidic isolation between both chamber sides.

4. Collection of Nerve organoids and Capsid sequences rescued by PCR
a. After 7 days, Nerve organoids were dissected manually by cutting through the microfluidic device with a razor blade.
b. After collecting spheroids, DNA was extracted from them using a miniprep DNA kit.
c. Capsid sequences that managed to infect the nerve organoids were recovered by PCR using specific capsid primers.
d. The PCR product was cloned back into the library plasmid and packaged into an AAV.
e. Capsid sequences harvested from neurons are analyzed using bioinformatics for conserved regions and highly enriched capsids can be de novo synthesised and can then undergo either further mutagenesis or repeat of in vitro screening to increase evolutionary pressure through directed evolution.
f. Directed evolution can be repeated for several rounds (^{~}2-5 rounds).

### Example 2-AAV Capsid Screening Via Direct Injection of AAV Library to Organoid Compartment

1. hiPS Cell Culture and differentiation
a. The hiPS cells were cultured on a Vitronectin-coated dish with Essential 8 Medium to maintain the undifferentiated state.
b. hiPS cells were grown for at least three passages and after they reached 70% confluency they were dissociated into single cells using accutase. Single cells were plated on p35 dishes coated with geltrex.
c. The first day the cells were plated in neurobasal medium supplemented with Rock inhibitor, CHIR99021, and bFGF. The next day Rock inhibitor was removed and the cells were maintained in NB medium supplemented with 3 uM CHIR 99021 and bFGF until day 3. The medium was changed every day.

2. Generation of neuromuscular organoids in 3D
a. Neuromesodermal progenitors generated from human PSCs at the previous step were dissociated using accutase to generate a single cell suspension.
b. On day 0 of organoid formation, NMP cells were plated on an ultra-low binding 96-well plate in Neurobasal medium with Rho-associated protein kinase ROCK inhibitor, bFGF and IGF1 and HGF.
c. After day 4 the organoids were maintained in NB medium without the addition of growth factors.
d. On day 10 the organoids were transferred in 60 mm dishes in 5 mL of medium and after 1 month in 100 mm dishes with 12 mL NB medium. During the whole period organoids were maintained on an orbital shaker rotating at 75 rpm.

3. AAV Capsid Libraries addition
a. AAV libraries were microinjected at the muscle side of the neuromuscular organoid.
b. Organoids were maintained on an orbital shaker for 7 days.

2. Collection of Nerve organoids and Capsid sequences rescued by PCR
a. After 7 days, Nerve organoids were micro dissected manually by cutting through the organoid with a micro scalpel.
b. After collecting the neuronal side, DNA was extracted from them using a miniprep DNA kit.
c. Capsid sequences that managed to infect the nerve organoids were recovered by PCR using specific capsid primers.
d. The PCR product was cloned back into the library plasmid and packaged into an AAV.
e. Capsid sequences harvested from neurons are analyzed using bioinformatics for conserved regions and highly enriched capsids can be de novo synthesised and can then undergo either further mutagenesis or repeat of in vitro screening to increase evolutionary pressure through directed evolution.
f. Directed evolution can be repeated for several rounds (^{~}2-5 rounds).

1. A method of screening for capsid-encoding nucleotide sequences of AAV viral particles capable of infecting population-specific neurons, the method comprising:
(i) obtaining or having obtained a neuromuscular organoid comprising neurons and muscle cells, wherein the neurons, having a cell body and a distal portion located away from the cell body, are disposed in the organoid such that the cell body and the distal portion are distally separated from each other;
(ii) contacting the distal portion of the neurons with a population of AAV viral particles such that they can infect the neuron at the distal portion;
(iii) recovering AAV viral particles that have infected the distal portion of the neurons from the cell body; and
(iv) determining the capsid encoding nucleotide sequences of the AAV viral particles recovered from the cell body.

2. A method of directed evolution to select for AAV viral particles capable of selectively infecting population-specific neurons comprising the steps:
(i) obtaining or having obtained a neuromuscular organoid comprising neurons and muscle cells, wherein the neurons, having a cell body and a distal portion located away from the cell body, are disposed in the organoid such that the cell body and the distal portion are distally separated from each other;
(ii) contacting the distal portion of the neurons with a population of AAV viral particles such that they can infect the neuron at the distal portion;
(iii) recovering AAV viral particles that have infected the distal portion of the neurons from the cell body;
(iv) determining the capsid encoding nucleotide sequences of the AAV viral particles recovered from the cell body, and
(v) optionally using the output of step (iv) to generate a new AAV viral particle library and repeating the method.

3.-11 (canceled)

12. The method according to claim 1 wherein the neuromuscular organoid is derived from iPSCs or ESCs.

13. The method according to claim 2 wherein the neuromuscular organoid is derived from iPSCs or ESCs.

14. The method according to claim 1 wherein the population-specific neurons are selected from the list consisting: mammalian, human, human sub-population and human disease-specific.

15. The method according to claim 14 wherein the population-specific neurons are selected from the list consisting: mammalian, human, human sub-population and human disease-specific.

16. The method according to claim 14 wherein the human disease-specific population-specific neurons are selected from the list: MND, DM, ALS, HD, epilepsy, neuropathy, PD, SCA, HSP, PLS, SMA, SBMA and LCCS neurons.

17. The method according to claim 15 wherein the human disease-specific population-specific neurons are selected from the list: MND, DM, ALS, HD, epilepsy, neuropathy, PD, SCA, HSP, PLS, SMA, SBMA and LCCS neurons.

18. AAV viral particles capable of infecting population-specific neurons identified or selectively evolved according to the method of any preceding claim.

19. Use of the AAV viral particles according to claim 18 to selectively infect population-specific neurons.

20. The use according to claim 19 wherein the AAV viral particle comprises a nucleotide-encoded payload.

21. The use according to claim 20 wherein the nucleotide-encoded payload encodes a therapeutic peptide.

22. Use of the AAV viral particles according to claim 19 in the treatment of a disease caused by a genetic mutation.
